(19) United States
(12) Patent Application Publication
(10) Pub. No.: US 2005/0043271 A1
(43) Pub. Date: Feb. 24, 2005

(54) Heparin-containing ophthalmic agent

(76) Inventors: Dorothea Gross, Ingbert (DE); Frank Holzer, Ingbert (DE)

Correspondence Address: Raymond J Lillie;Carella,Bymr Bain Gilfillan Cechi StewarT& Olstein, 5 Becker farm road, Roseland, NJ, 07068, US

(21) Appl. No.: 10/496,330

(22) PCT Filed: Feb. 11, 2002
(86) PCT No.: PCT/DE02/04526

(30) Foreign Application Priority Data
Dec. 12, 2001 (DE) 101-61-149.8

(51) Int. Cl. A61K 31/727; A61K 31/728; A61K 9/14

(52) U.S. Cl. 514/54; 514/56; 424/486

(57) ABSTRACT

The invention concerns the use of at least one pharmacologically compatible viscosity regulator and at least one mucopolysaccharide with heparin activity and optionally additional pharmaceutical auxiliary agents for the production of a pharmaceutical composition for the treatment of dry eyes or for the treatment of an allergically induced irritation of the eye.

The invention concerns the use of active substances for the production of a pharmaceutical composition for the treatment of ophthalmological malfunctions.

DE 195 47 972 A1 discloses the use of heparin or its pharmacologically compatible salts for prophylaxis and acute therapy in respect of allergic conjunctivitis. In particular in that case heparin is used in the form of eye drops or an eye spray with or without adjuvants or preserving agents.

Heparin is further used for the treatment of cauterisation of or burns as well as further injuries to the cornea of the eye, in which rapid healing of the wound to the cornea is required.

The exact active mechanism of heparin which in systemic use is employed primarily as an anticoagulant is unexplained. Recent research results point to interactions of the heparin with epithelial growth factors (Denk, P. O. et al., Invest. Ophthalmol. Vis. Sci. (1996) 37, 1005 and Knorr, M. et al., Ophthalmologe (1996) 93, 275). In addition modulation of the immune system and the occurrence of inflammation by heparin is discussed (Bowler, S. D. et al., Am. Rev. Respir. Dis. (1993) 147, 160).

In addition knowledge about an antiallergic action of heparin has been available for some years. In accordance therewith heparin inhibits degranulation, caused by antigens, of mast cells without direct antihistamine effect. That effect was observed both in relation to inhalative and also after intravenous application (Bowler, S. D. et al., Am. Rev. Respir. Dis. (1993) 147, 160; Ahmed, T. et al., N. Engl. J. Med. (1993) 329, 2: 90; Lucio, J. et al., J. Appl. Physiol. (1992) 73, 1093). It is also known from pharmacological experiments on animals that high-dosage heparin after topical application to the eye was found to be effective for the treatment of allergically caused conjunctival inflammation (Anderson, W. et al., Invest. Ophthalmol. Vis. Sci. (1994) 35, 1291).

The application of heparin to the eye is suitable to avoid or limit allergic overreaction on the part of the immune system. In accordance with recent knowledge allergically caused irritation of the eye is associated with the “dry eye” syndrome.

That “dry eye” syndrome is also referred to as the Sicca syndrome or also as the Sicca symptoms. The “dry eye” syndrome, the symptoms of which involve inter alia burning, scratchiness, and a gritty feeling in the eye as well as blurred vision is to be attributed to functional disturbances in the tear film. The cause of those functional disturbances is also for example environmental influences which give rise to allergies, such as for example pollution or the effect of ozone. In particular ozone pollution which rises in Summer months can not only give rise to a disturbance in tear production but can also cause destruction of individual constituents of the tear film. For example hyaluronic acid and proteins contained in natural tear film are destroyed by the effect of ozone. It has further been found that the Sicca syndrome is frequently linked to a contact allergy caused by cosmetics on the eye.

It is known from Spektrum Augenheilkunde (1998) 3/4: 174-176 that hypoosmolar sodium hyaluronate drops can be used for therapy for the “dry eye” syndrome. In that case sodium hyaluronate of a molecular weight of 5,000,000 Daltons was used.

It is further known from Spektrum Augenheilkunde (1995) 9/5: 215-217 that bacterially synthesised hyaluronate can be used for treating the “dry eye” syndrome. It is known from Jpn. J. Ophthalmol. (1996) 40: 62-65 that the improvement in tear film stability by means of sodium hyaluronate eye drops is dependent on dose.

The object of the present invention is to provide a use of active substances for the production of a pharmaceutical composition, which permits better therapy of allergically produced inflammation of the eye.

That object is attained by the use of at least one pharmacologically compatible viscosity regulator and at least one mucopolysaccharide with heparin activity and optionally additional pharmaceutical auxiliary agents for producing a pharmaceutical composition for the treatment of dry eyes or for the treatment of an allergically produced irritation of the eye.

The term “pharmacologically compatible viscosity regulator” is used in accordance with the invention to denote a viscosity regulator which is completely harmless from the toxicological and pharmacological point of view and which can thus be used for therapy on a human being, such as for example the human eye.

The term “viscosity regulator” is used in accordance with the invention to denote a compound or a composition with which for example, after introduction of the pharmaceutical composition into an aqueous solution, it is possible to set a desired viscosity. The viscosity regular preferably provides that the pharmaceutical composition which is present for example in fluid, gel-like or pasty form has a viscoelastic behaviour. The term viscoelastic behaviour is used in accordance with the invention to denote that the viscosity changes under the effect of compression, tension, thrust and/or shear stresses. In accordance with a preferred embodiment the pharmaceutical composition according to the invention which is present for example in fluid, gel-like or pasty form has the behaviour of a non-Newtonian fluid, by virtue of the viscosity regulator.

The term “mucopolysaccharide with heparin activity” is used to denote any mucopolysaccharide or glycosaminoglycan which has a biological or physiological activity comparable to heparin. By way of example a mucopolysaccharide with heparin activity, in relation to mast cells, causes a comparable biological effect insofar as for example degranulation of mast cells, caused by an antigen, is attenuated or inhibited. In that respect the mucopolysaccharide with heparin activity can have a higher or lower level of activity than the body-specific heparin of a patient. A desired biological or physiological activity can be implemented by adjusting the content of mucopolysaccharide in the pharmaceutical composition according to the invention.

The term “pharmaceutical auxiliary agents” is used to denote solvents, dissolving aids, dissolving accelerators, salt-forming agents, salts, buffer substances, viscosity- and consistency-influencing agents, gel-forming agents, emulsifiers, solubilisers, wetting agents, spreading agents, antioxidants, preserving agents, filling and carrier substances and so forth.

Preferably the pharmaceutical composition produced in accordance with the use of the invention is employed in the form of eye drops, eye solutions, eye lotions, eye sprays, eye ointments or eye tablets.

To produce an eye ointment the pharmacologically compatible viscosity regulator and the at least one mucopolysaccharide with heparin activity can be introduced for example into a mixture of viscous paraffin and white Vaseline. In addition low-viscosity paraffin or wool wax can also be used in ointments.

Preferably the pharmaceutical composition is prepared in the form of an eye spray or in the form of eye drops. In that respect generally the viscosity regulator and the mucopolysaccharide with heparin activity are dissolved in aqueous solutions.

In that respect, in accordance with a preferred embodiment, the aqueous solutions are isotonic solutions, with respect to the tear fluid. In the case of isotonic solutions osmolarity is approximately 300 mOsm/l. In accordance with a further preferred embodiment the pharmaceutical composition according to the invention is hypoosmolar. In that case osmolarity can be for example about 160-180 mOsm/l. A hypoosmolar solution is used in particular when an abnormally high level of osmolarity of a tear film in a patient with dry eyes has to be compensated.

Sodium chloride, boric acid etc. are used for isotonisation of the aqueous solution. The pH-value of the aqueous solution is in a range of pH 6 to 9, preferably pH 7.4 to 7.7. Buffer solutions such as for example phosphate buffer, acetate buffer, acetate-borate buffer, citrate buffer and borate buffer are used to adjust the pH-value.

In accordance with a preferred development the viscosity regulator is selected from the group which consists of hyaluronic acid, hyaluronate, chondroitin sulphate, polyvinyl pyrrolidone, polyvinyl alcohol, polyacrylic acid, polyacrylamide, polyacrylic resins, polyethylene glycol, cellulose derivatives and mixtures thereof.

For example hydroxyethylcellulose, hydroxypropylmethylcellulose and carboxymethylcellulose can be used as cellulose derivatives.

It has been found that the above-mentioned viscosity regulators have an irritation-reducing effect on the eye and have a lubrication action. The lubrication action delays the draining away of the pharmaceutical composition applied to the eye and thus prolongs the contact with the cornea of the eye. Accordingly the at least one mucopolysaccharide with heparin activity can be kept on the cornea over a longer period of time, for example at least 30 minutes to at least 60 minutes. The at least one mucopolysaccharide with heparin activity can thus act on the eye reliably over a long period of time when dealing with an allergically induced irritation of the eye, and can provide therapy for the inflammation process.

Caused by the viscoelastic properties of the pharmacologically compatible viscosity regulator, particularly when using hyaluronic acid or hyaluronate as the viscosity regulator, the mucopolysaccharide with heparin activity is readily distributed again substantially uniformly over the entire surface of the eye in each eyelid blink, insofar as a certain draining-away effect should occur.

The symptoms of an allergically induced irritation of the eye are closely related to the Sicca syndrome, that is to say the “dry eye” syndrome. The Sicca syndrome involves inadequate tear film formation. Both allergic irritation of the eye and also the Sicca syndrome involve reddening of the conjunctiva, eyelid swelling and itching, that is to say comparable inflammation symptoms.

The pharmaceutical composition produced in the use according to the invention reduces or prevents the liberation or the effect of histamine and further allergy mediators. The at least one mucopolysaccharide with heparin activity thus has for example an anti-allergy and itch-relieving action. The pharmaceutical composition with a viscosity which is adjusted by the viscosity regulator thus further acts as a slip or lubricating agent and thus represents a replacement for the inadequate tear film which occurs with Sicca syndrome. The itch which occurs due to mechanical irritation in the Sicca syndrome and reddening of the eye are reliably reduced by the slip or lubricating effect when using the pharmaceutical composition produced in accordance with the use of the invention.

The simultaneous action of mucopolysaccharide with heparin activity and the production of a synthetic tear film results in a synergistic action which permits faster therapy of an allergically induced irritation of the eye. As the eye is one of the most important sense organs for the human being, the pharmaceutical composition produced in accordance with the use of the invention represents a significant advance in the field of ophthalmology.

In accordance with a further preferred embodiment the amount of hyaluronic acid and/or the amount of hyaluronate is about 0.005% by weight to about 5% by weight, preferably about 0.01% by weight to about 1% by weight, in each case with respect to the total weight of the pharmaceutical composition.

Preferably, hyaluronic acid and/or hyaluronate is used as a pharmacologically compatible viscosity regulator in the pharmaceutical composition produced in the use according to the invention.

Hyaluronic acid is a constituent part of the vitreous humour of the eye and in that respect does not represent a compound which is foreign to the human organism. For that reason hyaluronic acid is very well compatible, from the immunological point of view. In addition hyaluronic acid or hyaluronate enjoys structural similarity with mucin. Mucin forms the lowermost layer of the three-layer tear film and provides for optimum wetting of the cornea and conjunctiva epithelia.

In addition hyaluronic acid has an excellent property for use on the eye, namely the viscosity rises with increasing shearing rate.

After application of the pharmaceutical composition produced by the use according to the invention to the cornea of the eye, a shearing stress is applied to the pharmaceutical composition by way of the blinking movement of the eyelid, whereby the initially increased viscosity is reduced. The viscosity is reduced due to the blinking movement of the eyelid so that a uniform film is formed on the surface of the eye. After the blink the viscosity increases so that the film adheres firmly to the surface of the eye.

Hyaluronic acid or salts thereof, hyaluronates, in particular sodium hyaluronate, has or have excellent optical properties so that there is no adverse effect on vision in the patients being treated.

The use of hyaluronic acid or hyaluronates in the pharmaceutical composition produced in accordance with the use of this invention is extremely advantageous in particular in terms of disturbance to wetting of the eye, that is to say in the case of what is referred to as “dry eye”, and for the treatment of epithelium lesions which result from disturbances to wetting of the eye.

When using hyaluronic acid and/or hyaluronate in the pharmaceutical composition the pharmaceutical composition is preferably prepared free from preserving agent.

Preserving agents can damage the pre-corneal tear film and lead to a reduction in the number of microvilli and microplicae of the surface cornea epithelium cells. In particular the wide-spread benzalkonium chloride has a great damage potential. In regard to the desired therapy of an allergically induced irritation of the eye or irritation induced by the Sicca syndrome, any further irritation and/or damage to the eye by the addition of preserving agents is to be avoided.

Preferably for storage and delivery of a preserving agent-free, pharmaceutical composition produced in accordance with the use of this invention, use is made of the Comod® system described in “PTA heute” 1996, No 12, pages 1230-1232, which permits sterile storage and multiple delivery of the specified pharmaceutical composition. It will be appreciated that it is also possible to use conventional single-dose containers which are thrown away after use.

Particularly preferably the amount of hyaluronic acid and/or the amount of hyaluronate is about 0.05% by weight to about 0.5% by weight.

Extremely advantageously hyaluronic acid and hyaluronates respectively has or have the property of binding water. That property of binding water is particularly advantageous in regard to treatment of the Sicca syndrome as unwanted drying-out of the cornea of the eye is counteracted. Levels of concentration of 0.1% by weight to 0.3% by weight have proven to be highly satisfactory.

A further diagnostic parameter in regard to diagnosis of the “dry eye” syndrome is the tear film tearing time which makes it possible to provide information about the quality of the tear fluid. In that case for example the tear film is dyed with fluorescein and the patient is then asked to keep the eyes open as long as possible without a blink reflex. A slit lamp is then used to establish when the tear film tears open for the first time. If the period of time is less than 10 seconds, there is the suspicion of the “dry eye” syndrome. In that respect hyaluronic acid at a concentration of 0.1% by weight to 0.3% by weight has proven to be highly effective in regard to prolonging the tear film tearing time.

The hyaluronic acid or the hyaluronate can be isolated from the vitreous humour of a bovine eye but also from cockscombs. In addition hyaluronic acid or hyaluronate can also be produced in bacterial strains in pharmaceutical quality.

For example potassium, sodium, calcium and/or magnesium hyaluronates can be used as salts of hyaluronic acid.

The hyaluronate sodium hyaluronate is particularly preferred.

Aqueous sodium hyaluronate solutions are fluids with non-Newtonian flow properties. By virtue of that physical property, aqueous sodium hyaluronate solutions are excellently well suited as slip and lubricating agents with a good cling effect and a prolonged residence time on the conjunctival and corneal epithelia, without adversely affecting visual efficiency. A concentration of 0.1% by weight of sodium hyaluronate in the composition produced by the use according to the invention considerably improves the subjective feeling of the patients, which is important when treating dry eyes.

In addition sodium hyaluronate-bearing eye drops exhibit properties such as to promote healing of wounds on the epithelia of the eye, in animal testing. It was found that hyaluronic acid or sodium hyaluronate, in dependence on concentration, promoted the migration of epithelium cells and thus wound healing. A 0.1% by weight sodium hyaluronate solution implemented increased epithelium cell migration in the case of cornea epithelium cells of rabbits.

Hyaluronic acid or sodium hyaluronate also caused faster and better wound healing, that is to say which takes place with less scarring, in the event of injury to the cornea epithelium or in the case of cauterisation of the cornea.

The precise operative mechanism involved in the promotion of wound healing by hyaluronic acid is still unexplained. While an influence on the circulation of blood through the surrounding cells appears to be less probable, there are various pointers to an effect on cells which play a part in the inflammation process.

Finally, in dependence on dose, hyaluronic acid exhibits a protective action in relation to damage to cells by oxygen radicals. Free oxygen radicals slow down the wound healing process and thus play a crucial role in the inflammation situation.

The anti-inflammatory effect of hyaluronic acid or hyaluronate and the protection afforded by hyaluronic acid or hyaluronate from the harmful effect of oxygen radicals co-operate in synergistic relationship with the anti-inflammatory action of the mucopolysaccharide with heparin activity in the pharmaceutical composition produced by the use in accordance with the invention.

The non-Newtonian flow properties of the pharmaceutical composition, which are further produced by hyaluronic acid or hyaluronate in prepared solutions, gels, pastes or ointments, besides an excellent slip and lubricating effect, also afford excellent adhesion to the cornea of the eye. Mechanical irritation of the eye which occurs with the Sicca syndrome is greatly reduced or eliminated. In addition, the fact that adhesion of the pharmaceutical composition on the cornea of the eye is improved by virtue of the anti-Newtonian flow properties provides for faster healing of the inflammatory processes.

In accordance with a further preferred embodiment the hyaluronic acid and/or hyaluronate are of a molecular weight which is in a range of about 50,000 to about 10,000,000 Daltons, preferably from about 250,000 to about 5,000,000 Daltons. Particularly preferably the molecular weight of the hyaluronic acid or the hyaluronate is from 500,000 to 4,000,000 Daltons. Very preferably the hyaluronic acid or the hyaluronate is of a molecular weight of about 1,500,000 to 3,500,000 Daltons.

The high molecular weight of the hyaluronic acid or the hyaluronate used such as for example sodium hyaluronate provides for a high level of viscoelasticity at a low level of concentration. The molecule chains are present in the solution in a random arrangement in a tangled configuration. Under the influence of the shearing forces exerted by the movement of the eyelid, the macromolecules are oriented in substantially parallel relationship. That change in the three-dimensional structure under the influence of shearing forces is thought to be crucial for the excellent viscoelastic properties.

Upon lid opening the substance covers over the surface of the cornea and, by virtue of the high water binding capacity of hyaluronate, also represents protection against evaporation. That is advantageous in particular in relation to the dry eye syndrome which involves a reduction in the amount of tear fluid in the eye.

It is further preferred if the heparin activity of the mucopolysaccharide is in a range of about 200 I.U./ml to about 100,000 I.U./ml, preferably about 1,000 I.U./ml to about 50,000 I.U./ml. Preferably the heparin activity is in a range of from about 1,200 I.U./ml to about 10,000 I.U./ml. Very preferably the heparin activity is in a range of from about 1,300 I.U./ml to about 5,000 I.U./ml.

In that respect heparin activity is determined in accordance with the Pharmacopoea Europaea 1997. The unit “I.U./ml” is an abbreviation for “International Unit/ml”.

The heparin activity of the mucopolysaccharide can be determined by way of any suitable test for determining heparin activity. What is essential is that the comparison of the activity of human heparin and the heparin activity of the mucopolysaccharide is effected with the same test under comparable test conditions.

In accordance with a further preferred embodiment the mucopolysaccharide with heparin activity is selected from the group which consists of heparinoids, human heparin, animal heparin, recombinant heparin, chemically modified heparin, enzymatically modified heparin, truncated heparin, low-molecular heparin, heparan sulphate and mixtures thereof.

The molecular weight of heparin is usually in a range of about 5,000 to about 30,000 g/mol, preferably about 6,000 to about 20,000 g/mol.

Low-molecular heparin generally involves a molecular weight in a range of about 4,000 to about 8,000 g/mol and can be obtained from natural heparin by depolymerisation, for example using nitric acid.

It will be appreciated that all of the above-mentioned mucopolysaccharides with heparin activity can also be used in the form of the respective pharmacologically compatible salts in the pharmaceutical composition produced by the use according to the invention.

The precise dosage of the heparin activity in the pharmaceutical composition varies in dependence on the allergically induced irritation of the eye, which is to be treated.

It is further preferred if the pharmaceutical composition is in the form of a solution, suspension, emulsion, gel, ointment, paste, powder, granulate or tablet. The pharmaceutical composition is preferably an ophthalmic agent, further preferably a heparin-bearing ophthalmic agent.

In accordance with a preferred embodiment the pharmaceutical composition is in the form of a solution so that it can be applied to the surface of the cornea of the eye for example in the form of eye drops or an eye spray.

It will be appreciated that it is possible for the pharmaceutical composition produced by the use according to the invention to be in the form of a solid which prior to application is firstly dissolved in an aqueous solution such as for example a buffer solution. After dissolution of a solid, for example in an aqueous buffer solution, that solution is subjected to sterile filtering and then applied to the cornea as an eye spray or eye drops. Preferably, the solid and the solvent are already in sterile form when stored separately so that sterile filtration after production of the solution is not required. Thus, the user can apply the pharmaceutical composition directly after making up the mixture or solution.

When preparing the pharmaceutical composition in the form of a solid, such as for example a powder, particles, granules or a tablet the pharmaceutical composition produced by the use according to this invention preferably includes heparin sodium and/or heparin potassium as well as hyaluronic acid and/or sodium hyaluronate, as those compounds are very soluble in water. Prior to application then for example heparin sodium and hyaluronic acid are mixed together in the desired quantitative ratios and dissolved with the addition of water or aqueous buffer solutions and then subjected to sterile filtration.

In principle the pharmaceutical composition produced by the use according to the invention can also be introduced into the conjunctival sac in the form of eye tablets. The eye tablet quickly dissolves under the action of tear fluid.

However application of the pharmaceutical composition in the form of eye drops or an eye spray is preferred.

When preparing the pharmaceutical composition in the form of eye ointments or eye gels, the active substance are prepared for example in Vaseline or paraffin with and without the addition of emulsifier such as for example cholesterol, wool wax, wool wax alchohols, cetanol, and so forth.

Particularly preferably the pharmaceutical composition produced in accordance with the use of this invention as set forth in one of claims 1 to 8 is used in the treatment of ophthalmological malfunctions, which are selected from the group consisting of the Sicca syndrome, allergic rhinoconjunctivitis, atopic keratoconjunctivitis, allergic keratoconjunctivitis, gigantopapillary conjunctivitis, conjunctivitis vernalis, episcleritis such as for example episcleritis periodica, episcleritis partialis fugax, scleritis, tenonitis, Sjögren syndrome and hybrid forms thereof.

### EXAMPLE 1

2,000 I.U. of heparin sodium

1.0 mg of sodium hyaluronate, molecular weight: 1.5×10⁶−3.5×10⁶ Daltons

2.0 mg of sodium dihydrogenphosphate dihydrate

12.0 mg of disodium hydrogenphosphate dodecahydrate

27.0 mg of sorbitol

ad 1.0 ml of water for injection purposes

### EXAMPLE 2

4,000 I.U. of heparin sodium

1.0 mg of sodium hyaluronate, molecular weight: 1.5×10⁶−3.5×10⁶ Daltons

2.0 mg of sodium dihydrogenphosphate

12.0 mg of disodium hydrogenphosphate dodecahydrate

27.0 mg of sorbitol

ad 1.0 ml of water for injection purposes

1. Use of at least one pharmacologically compatible viscosity regulator and at least one mucopolysaccharide with heparin activity and optionally additional pharmaceutical auxiliary agents for the production of a pharmaceutical composition for the treatment of dry eyes or for the treatment of allergically induced irritation of the eye.

2. Use according to claim 1 characterised in that the viscosity regulator is selected from the group consisting of hyaluronic acid, hyaluronate, chondroitin sulphate, polyvinyl pyrrolidone, polyvinyl alcohol, polyacrylamide, polyacrylic resins, polyethylene glycol, cellulose derivatives and mixtures thereof.

3. Use according to claim 2 characterised in that the amount of hyaluronic acid and/or the amount of hyaluronate is about 0.005% by weight to about 5% by weight, preferably about 0.01% by weight to about 1% by weight, in each case with respect to the total weight of the pharmaceutical composition.

4. Use according to claim 2 or claim 3 characterised in that the hyaluronic acid and/or the hyaluronate has a molecular weight which is in a range of about 50,000 to about 10,000,000 Daltons, preferably about 250,000 to about 5,000,000 Daltons.

5. Use according to one of claims 2 to 4 characterised in that the hyaluronate is sodium hyaluronate.

6. Use according to one of claims 1 to 5 characterised in that the heparin activity of the mucopolysaccharide is in a range of about 500 I.U./ml to about 100,000 I.U./ml, preferably about 1,000 I.U./ml to about 50,000 I.U./ml.

7. Use according to one of claims 1 to 6 characterised in that the mucopolysaccharide is selected from the group which consists of heparinoids, human heparin, animal heparin, recombinant heparin, chemically modified heparin, enzymatically modified heparin, truncated heparin, low-molecular heparin, heparan sulphate and mixtures thereof.

8. Use according to one of claims 1 to 7 characterised in that the pharmaceutical composition is in the form of a solution, suspension, emulsion, gel, ointment, paste, powder, particles, granules or a tablet.

9. Use according to one of claims 1 to 8 characterised in that the dry eyes or the allergically induced irritation of the eye are or is due to an ophthalmological malfunction selected from the group consisting of Sicca syndrome, allergic rhinoconjunctivitis, atopic keratoconjunctivitis, allergic keratoconjunctivitis, gigantopapillary conjunctivitis, conjunctivitis vernalis, episcleritis, scleritis, tenonitis, Sjögren syndrome and hybrid forms thereof.
